(19) United States
(12) Patent Application Publication
(10) Pub. No.: US 2026/0248977 A1
(43) Pub. Date: Aug. 27, 2026

(54) Method for Reducing Germ Density, and Corresponding Device

(71) Applicants: Thermo Electron LED GmbH, Langenselbold (DE); Thermo Orion Inc., Chelmsford, MA (US)

(72) Inventors: Waldemar PIECZAREK, Langenselbold (DE); Kannan SRINIVASAN, Chelmsford, MA (US); Jennifer D'ONOFRIO, Langenselbold (DE); Verena RUCKSTUHL, Langenselbold (DE)

(21) Appl. No.: 19/552,033

(22) Filed: Feb. 27, 2026

(30) Foreign Application Priority Data
Feb. 27, 2025 (DE) 102025107488.7

(51) Int. Cl. A61L 9/14; B01D 53/00; F24F 1/0087; F24F 1/009

(52) U.S. Cl. CPC A61L 9/14; B01D 53/005; F24F 1/0087; F24F 1/009; A61L 2209/111; A61L 2209/13; A61L 2209/211; B01D 2257/80; B01D 2258/06

(57) ABSTRACT

The invention relates to a method for reducing a germ density in the usable space of a device, comprising heating the usable space to a process temperature such that the relative humidity in the usable space is below an upper humidity limit value; regulating the relative humidity in the usable space to a first target value for a first duration, wherein a humidification system introduces an active substance; deactivating the relative humidity regulation; and degrading the active substance in the usable space for a degradation duration. The invention also relates to a corresponding device and to a corresponding system.

### CROSS REFERENCE TO RELATED APPLICATIONS

This U.S. Nonprovisional Patent Application claims priority to German Patent Application No. DE102025107488.7 filed Feb. 27, 2025, the contents of which are incorporated herein for any and all purposes.

### TECHNICAL FIELD

The present invention relates to the field of reducing germ contamination in usable spaces in devices, e.g. laboratory, climate control and heating devices, and in particular sterilizing the usable spaces.

### BACKGROUND

The usable spaces of devices, in particular laboratory, climate control and heating devices, must be as sterile as possible for certain uses, e.g. for cell culture and cell therapy applications. There are also objects, e.g. certain surgical devices or, more generally, certain medical devices, that must be sterilized once or regularly. Therefore, it is generally necessary to have a means of reducing the germ density and, preferably, sterilizing the usable space of such devices in order to reduce or, advantageously, eliminate contamination by bacteria, viruses, mold, or other undesirable organisms in the usable space and/or objects contained therein.

One generally known and applied method is to reduce the germ density by increasing the temperature. However, this can have several disadvantages. On the one hand, this can be a lengthy and energy-intensive process; on the other hand, there are materials that cannot be exposed to such high temperatures without being damaged.

Sterilization methods that use a sterilizing agent are known as an alternative. Due to its chemical properties, hydrogen peroxide (H₂O₂) is particularly suitable for this purpose. However, hydrogen peroxide is highly corrosive and cytotoxic, so care must be taken when handling this sterilizing agent to ensure that it is not released in order to avoid damage to users and the environment.

For example, hydrogen peroxide sterilization chambers for medical devices are known, in which highly concentrated hydrogen peroxide is used, e.g. in a vacuum or in a plasma, and then rendered harmless. However, these are purely sterilization chambers and not laboratory, climate control or heating devices, such as incubators. In addition, these sterilization chambers typically have a small volume.

Mobile generators are also known, which can be used, for example, to decontaminate rooms, isolators, or surfaces and typically vaporize or nebulize the hydrogen peroxide. However, these generators typically do not have regulated processes and are therefore not suitable for changing ambient conditions. Furthermore, such generators are designed for the decontamination of entire laboratory rooms and are associated with high costs.

In addition, sterilization devices for installation in corresponding laboratory, climate control or heating devices are known. For example, US 2022/0243166 A1 discloses an atomization device that atomizes a hydrogen peroxide solution by means of a diaphragm. However, this can leave behind residues in the usable space that are hazardous to the user, and the device in question requires installation and removal.

The ReCO2ver™ incubator by Baker offers the option of using the internal nebulizer to decontaminate the interior of the incubator. To do this, a user first warms up a hydrogen peroxide solution and then manually fills it into the reservoir of the nebulizer. The interior is then heated to 45° C. before the nebulizer runs for around 20 minutes. A UV light is then switched on for approximately 130 minutes to decompose the suspended hydrogen peroxide. The user then empties the reservoir manually and cleans it. This procedure has the disadvantage that the user must manually fill, remove, and dispose of the hydrogen peroxide solution, as well as clean the reservoir afterward. This poses the risk of the user coming into direct contact with the hydrogen peroxide solution. Furthermore, the nebulization of the hydrogen peroxide solution is not regulated, but only controlled by the duration.

### SUMMARY

Against this background, the object of the present invention is to overcome or at least reduce the shortcomings and drawbacks of the prior art. In general, the object of the present invention may be to provide a device with an integrated means of reducing the germ density and preferably sterilizing the usable space.

This object is achieved by the subjects of the independent claims. Advantageous further developments of the invention are described by the dependent claims, the following description, and the figures.

### BRIEF DESCRIPTION OF THE DRAWINGS

Embodiments of the present invention will now be described with reference to the accompanying drawings. These embodiments are only intended to exemplify, but not limit, the present invention.

FIG. 1 shows a device comprising a usable space, a humidification system, and a temperature control system;

FIG. 2 shows an embodiment of a device according to the present invention;

FIG. 3 shows an embodiment of a method for reducing germ density;

FIG. 4 shows another embodiment of a method for reducing germ density; and

FIG. 5 schematically illustrates a curve of temperature and relative humidity during implementation of the method for reducing germ density.

It is pointed out that not all drawings bear all reference signs. Instead, in some of the drawings, some of the reference signs have been omitted for brevity and ease of presentation. Embodiments of the present invention will now be described with reference to the accompanying drawings.

### DETAILED DESCRIPTION OF ILLUSTRATIVE EMBODIMENTS

According to a first aspect, the invention relates to a method for reducing a germ density in the usable space of a device, comprising heating the usable space to a process temperature such that a relative humidity in the usable space is below an upper humidity limit value; regulating the relative humidity in the usable space to a first target value for a first duration, wherein a humidification system introduces an active substance; deactivating the relative humidity regulation; and degrading the active substance in the usable space for a degradation duration.

This means that the relative humidity in the usable space is regulated to a first target value for a first duration, while a humidification system introduces an active substance, for example by vaporizing or nebulizing (in particular atomizing) the latter.

An active substance is therefore introduced into the usable space. This active substance can reduce the germ density. For example, the active substance may be hydrogen peroxide H₂0₂. During the introduction of the active substance, the relative humidity in the usable space is regulated to a first target value. Later, the active substance in the usable space is degraded for a degradation duration.

A suitable reduction in germ density, i.e. for example a suitable sterilization, can thus be achieved. In particular, the invention makes it possible to achieve a suitable reduction in germ density with relatively few adjustments to the equipment: In particular, a humidification system, i.e. a system that is already present in many devices (e.g. in incubators) is used to introduce the active substance. By embodiments according to the invention, it is in particular also possible to achieve the situation where the reduction in germ density is achieved at relatively low temperatures (e.g. 55° C. or below). This may be advantageous for various reasons: It can shorten the duration of the method, the energy required can be relatively low, and there are fewer requirements placed on the materials than would be the case at higher temperatures (e.g. 180° C.).

In embodiments of the invention, it may be provided that the method further comprises: inserting an active substance container containing an active substance liquid, and inserting a waste container for collecting waste liquids.

In embodiments of the invention, it may be provided that the method further comprises: introducing a degradation unit into the usable space of the device.

In embodiments of the invention, it may be provided that the degradation unit is introduced into the usable space before heating.

In embodiments of the invention, it may be provided that introducing the degradation unit into the usable space comprises electrically connecting the degradation unit to the device.

In embodiments of the invention, it may be provided that the degradation unit is designed to decompose the active substance.

In embodiments of the invention, it may be provided that heating the usable space to the process temperature comprises increasing the process temperature until the relative humidity at the process temperature is below the upper humidity limit value.

In embodiments of the invention, it may be provided that heating the usable space to the process temperature comprises heating the usable space to an initial value for the process temperature, wherein the initial value is provided by the user.

In embodiments of the invention, it may be provided that the initial value is at least 37° C.

In embodiments of the invention, it may be provided that, during and/or after heating to the initial value, the relative humidity in the usable space is determined and is compared with the upper humidity limit value.

In embodiments of the invention, it may be provided that, if the relative humidity is not below the upper humidity limit value, the process temperature is increased until the relative humidity is below the upper humidity limit value.

In embodiments of the invention, it may be provided that the process temperature is increased in stages until it is below the upper humidity limit value.

In embodiments of the invention, it may be provided that increasing the process temperature comprises determining the process temperature based on the current relative humidity and temperature using the vapor pressure curve.

In embodiments of the invention, it may be provided that the process temperature is greater than or equal to the initial value and is selected such that the relative humidity at the process temperature is below the upper humidity limit value.

In embodiments of the invention, it may be provided that the process temperature is at least 37° C.

In embodiments of the invention, it may be provided that the process temperature is at most 75° C., preferably at most 65° C., more preferably at most 55° C.

In embodiments of the invention, it may be provided that the relative humidity is not regulated during heating.

In embodiments of the invention, it may be provided that the upper humidity limit value is in the range of 45% to 75% relative humidity, preferably in the range of 45% to 60% relative humidity, more preferably in the range of 45% to 55% relative humidity.

In embodiments of the invention, it may be provided that the process temperature after heating is kept constant at least until the active substance in the usable space has degraded.

In embodiments of the invention, it may be provided that the method further comprises: increasing the relative humidity in the usable space to a value above a lower humidity limit value.

In embodiments of the invention, it may be provided that the relative humidity in the usable space is increased to a value above a lower humidity limit value before the relative humidity is regulated to a first target value for a first duration.

In embodiments of the invention, it may be provided that the relative humidity in the usable space is increased to a value above a lower humidity limit value after the usable space has been heated to the process temperature.

In embodiments of the invention, it may be provided that increasing the relative humidity in the usable space to a value above a lower humidity limit value comprises vaporizing water.

In embodiments of the invention, it may be provided that the lower humidity limit value is in the range of 30% to 40% relative humidity.

In embodiments of the invention, it may be provided that the relative humidity in the usable space is increased to a value above a lower humidity limit value before the relative humidity in the usable space is regulated to a first target value.

In embodiments of the invention, it may be provided that the first target value is a first amount above the humidity at the start of regulating the relative humidity to a first target value.

In embodiments of the invention, it may be provided that the first amount is in the range of 10% to 40% relative humidity, more preferably in the range of 15% to 30% relative humidity, even more preferably in the range of 15% to 25% relative humidity.

In embodiments of the invention, it may be provided that the first duration is in the range of 5 min to 120 min, preferably in the range of 30 min to 90 min, more preferably in the range of 40 to 70 min.

In embodiments of the invention, it may be provided that introducing the active substance comprises vaporizing or nebulizing an active substance liquid in the humidifier of the humidification system.

In embodiments of the invention, it may be provided that the active substance liquid is provided in an active substance container and/or at an active substance port of the device.

In embodiments of the invention, it may be provided that the active substance liquid is an active substance solution.

In embodiments of the invention, it may be provided that the active substance is a sterilizing agent.

In embodiments of the invention, it may be provided that the active substance is hydrogen peroxide (H₂O₂).

In embodiments of the invention, it may be provided that the method further comprises: after the first duration has elapsed, determining the current relative humidity in the usable space and regulating the relative humidity to a second target value for a second duration, wherein the humidification system introduces an active substance liquid.

In embodiments of the invention, it may be provided that the second target value is a second amount above the current relative humidity.

In embodiments of the invention, it may be provided that the second amount is in the range of 10% to 40% relative humidity, more preferably in the range of 15% to 30% relative humidity, even more preferably in the range of 15% to 25% relative humidity.

In embodiments of the invention, it may be provided that the second duration is in the range of 5 min to 120 min, preferably in the range of 30 min to 90 min, more preferably in the range of 40 to 70 min.

In embodiments of the invention, it may be provided that the steps of deactivating the regulation, degrading the active substance, and flushing are only performed after the first and/or second duration has elapsed.

In embodiments of the invention, it may be provided that regulating the relative humidity in the usable space to a first target value and/or a second target value comprises actively distributing the air in the usable space of the device.

In embodiments of the invention, it may be provided that the method comprises diluting the active substance liquid prior to vaporization or nebulization.

In embodiments of the invention, it may be provided that degrading the active substance in the usable space comprises activating the neutralization unit.

In embodiments of the invention, it may be provided that degrading the active substance comprises decomposing the active substance.

In embodiments of the invention, it may be provided that the degradation duration is selected such that a residual concentration of the gaseous active substance in the usable space does not pose a hazard to a user.

In embodiments of the invention, it may be provided that the degradation duration is in a range of 10 min to 600 min, preferably in a range of 20 min to 400 min, more preferably in a range of 30 min to 240 min.

In embodiments of the invention, it may be provided that the method further comprises: flushing at least some of the supply pipes that supply to a humidifier of the humidification system.

In embodiments of the invention, it may be provided that flushing is performed bidirectionally.

In embodiments of the invention, it may be provided that flushing comprises emptying liquid pipes into a waste container.

In embodiments of the invention, it may be provided that flushing comprises filling the emptied liquid pipes with fresh water.

In embodiments of the invention, it may be provided that flushing comprises emptying the liquid pipes into the waste container again.

In embodiments of the invention, it may be provided that the emptying and/or filling is performed by means of a bidirectional pump.

In embodiments of the invention, it may be provided that the method further comprises: locking a door to the usable space of the device, wherein the locking is performed prior to heating the usable space to a process temperature.

In embodiments of the invention, it may be provided that the method further comprises: performing an automatic check of the device, wherein the check is performed prior to heating the usable space to a process temperature, and heating the usable space to a process temperature is only performed if the check is successful.

In embodiments of the invention, it may be provided that performing the automatic check comprises:
checking a temperature control system,
checking a humidity system,
checking a ventilation system, and/or
checking a water quantity in a storage tank.

In embodiments of the invention, it may be provided that performing the automatic check comprises:—checking a status of the degradation unit.

In embodiments of the invention, it may be provided that the humidification system comprises a liquid sensor, wherein performing the automatic check comprises: checking a status of the liquid sensor.

In embodiments of the invention, it may be provided that, at least during the first duration, an alarm system that monitors at least one function of the device is active.

Overall, different safety mechanisms may thus be provided. The door can be locked. It may also be provided that the process can only be started if there are no faults in the temperature, humidity, and ventilation systems and if the water reservoir is filled with a sufficient quantity of water. In addition, it can be checked whether the degradation unit (which can also be referred to as the neutralization unit) is connected. If a sensor is used to detect the water column (e.g. in a hose upstream of the vaporizer), the functioning thereof can also be checked. During the introduction of the active substance (which can also be referred to as fumigation, for example), the same alarm system that is used in a possible incubation mode can also be active at all times. It can have different functions, such as: failure of sensors, failure of the fan, failure of the heaters, monitoring of the water supply, and/or exceeding of setpoints of the controllers.

In embodiments of the invention, it may be provided that the device is a laboratory device.

In embodiments of the invention, it may be provided that the device is a climate control or heating device.

In embodiments of the invention, it may be provided that the device is an incubator, for example a CO₂ incubator and/or a reach-in incubator.

According to a further aspect, the invention is directed to a device. The device comprises: a usable space; an active humidification system designed to increase the relative humidity in the usable space; a humidity sensor for measuring the relative humidity in the usable space; a temperature control system designed to increase the temperature in the usable space; a temperature sensor for measuring the temperature in the usable space; wherein the humidification system comprises at least one active substance port designed for connection to an active substance container containing an active substance liquid comprising an active substance; and wherein the device is designed to introduce the active substance into the usable space via the humidification system.

In embodiments of the invention, it may be provided that the humidification system further comprises at least one waste port designed for connection to a waste container, wherein the device is designed to flush at least part of the humidification system and to direct resulting waste liquid to the waste port.

In embodiments of the invention, it may be provided that the humidification system comprises a humidifier designed to vaporize and/or nebulize liquids.

In embodiments of the invention, it may be provided that the humidifier is a vaporizer and/or a nebulizer.

In embodiments of the invention, it may be provided that the humidification system comprises a water port designed for connection to a water reservoir that provides fresh water.

In embodiments of the invention, it may be provided that the humidification system comprises a pump.

In embodiments of the invention, it may be provided that the pump is a bidirectional pump.

It will be understood that a bidirectional pump can pump in both directions.

In embodiments of the invention, it may be provided that the pump is designed to convey liquids from the active substance port to the humidifier.

In embodiments of the invention, it may be provided that the pump is designed to convey liquids from the water port to the humidifier.

In embodiments of the invention, it may be provided that the humidification system comprises a valve matrix designed to enable fluid connections between different ports.

In embodiments of the invention, it may be provided that the valve matrix is further designed to adjust the flow rate for at least one fluid connection.

In embodiments of the invention, it may be provided that the valve matrix is arranged between the pump and the active substance port.

In embodiments of the invention, it may be provided that the valve matrix is arranged between the pump and the water port.

In embodiments of the invention, it may be provided that the valve matrix is arranged between the pump and the waste port.

In embodiments of the invention, it may be provided that the valve matrix comprises at least 2 valves.

In embodiments of the invention, it may be provided that the device additionally comprises an air distribution system designed to circulate the air in the usable space.

In embodiments of the invention, it may be provided that the air distribution system comprises a fan.

In embodiments of the invention, it may be provided that the device further comprises a ventilation system designed to supply the usable space with supply air.

In embodiments of the invention, it may be provided that the device is designed to carry out the described method.

In embodiments of the invention, it may be provided that the device comprises a processing unit designed to control the device.

In embodiments of the invention, it may be provided that the processing unit is designed to carry out the described method.

In embodiments of the invention, it may be provided that the active substance liquid is an active substance solution.

In embodiments of the invention, it may be provided that the active substance is a sterilizing agent.

In embodiments of the invention, it may be provided that the active substance is hydrogen peroxide (H₂O₂).

In embodiments of the invention, it may be provided that the device is a laboratory device.

In embodiments of the invention, it may be provided that the device is a climate control or heating device.

In embodiments of the invention, it may be provided that the device is an incubator, for example a CO₂ incubator and/or a reach-in incubator.

In embodiments of the invention, it may be provided that the humidification system further comprises a liquid sensor arranged between the humidifier and the pump.

In embodiments of the invention, it may be provided that the device is a device as described above.

According to a further aspect, the invention relates to a system. The system comprises the device described and a degradation unit designed to degrade an active substance in the usable space of the device.

In embodiments of the invention, it may be provided that the degradation unit is designed to be placed in the usable space and electrically connected to the device.

In embodiments of the invention, it may be provided that the degradation unit comprises a UV lamp.

In embodiments of the invention, it may be provided that the degradation unit comprises a reaction partner and/or catalyst for the active substance.

In embodiments of the invention, it may be provided that the degradation unit comprises a fan designed to direct air over the reaction partner and/or catalyst.

In general, embodiments of the invention are therefore directed to a method, a device, and a system for reducing germ density.

It will be understood that a standard device (e.g. in an incubator) with an active humidification system typically includes the following components
water tank or external water connection
steam generator
pipe or hose system for hydraulic connections
pump or valve for metering the water
humidity sensor
system for distributing air in the usable space
heater(s), and
temperature sensor.

According to embodiments of the present invention, such a device or a corresponding system with the existing components can be extended to other tasks. The system can be extended such that other containers containing additional liquid media can be connected to the connected water container. Valves can be used to select from which container the liquid is metered to the vaporizer. Following vaporization from these other containers, any residues in the hydraulic system can be flushed out using water from the first container. Water container 1 remains connected to the system. po The residual liquids in the hydraulic system and the flushing liquid can be directed as waste liquid into a third container.

Embodiments of the invention thus comprise extending the humidification system to a sterilization system for the interior.

The following tasks can be performed by embodiments of the system according to the invention and by vaporizing appropriate sterilizing agents
reducing the germ load in the usable space,
disinfecting the usable space, and/or
sterilizing the usable space.

In addition, a valve unit can be connected in order to mix a different sterilizing agent from various starting chemicals or to produce a dilution solution from a concentrate.

The following advantages can be achieved by means of embodiments according to the invention:

The same, already present components that perform the standard humidification function can be used. This means that relatively few additional adjustments to the components are necessary.

An automated and regulated process can be implemented, and regulation can take place via the device (e.g. the incubator).

The process can be monitored and automatically adjusted to the ambient conditions at the installation site (the sterilization temperature is adjusted as a function of the relative humidity).

At least part of the water pipe system can be decontaminated with liquid sterilizing agent.

The process can be carried out at 37° C. or slightly higher temperatures (e.g. 50° C.), so that no cooling time (or only a relatively short cooling time) is necessary.

In general, the process can also be faster than sterilization routines that are carried out at 180° C., for example.

The insulation thickness of the device can be reduced compared to heat sterilization (e.g. at 180° C.).

The choice of materials for the overall system can be expanded, since 180° C. resistance has very high requirements, which is not necessary in embodiments of the invention.

It will be understood that it is possible to have lower surface temperatures on the device (e.g. incubator) during the process than in the case of sterilization at approx. 180° C.

Overall, it may also be more suitable for cleanroom applications than processes at 180° C. (no risk of increased particle release and disruption of cleanroom air flow due to thermal effects).

The invention is also defined by the following numbered embodiments.

Reference will be made below to method embodiments. These embodiments are indicated by an M followed by a number. When method embodiments or M-embodiments are mentioned below, these embodiments are meant.

M1. A method for reducing a germ density in the usable space of a device, comprising
heating the usable space to a process temperature such that the relative humidity in the usable space is below an upper humidity limit value;
regulating the relative humidity in the usable space to a first target
value for a first duration, wherein a humidification system introduces an active substance;
deactivating the relative humidity regulation; and
degrading the active substance in the usable space for a degradation duration.

This means that the relative humidity in the usable space is regulated to a first target value for a first duration, while a humidification system introduces an active substance, for example by vaporizing or nebulizing (in particular atomizing) the latter.

M2. The method according to the preceding method embodiment, wherein the method further comprises
inserting an active substance container containing an active substance liquid, and
inserting a waste container for collecting waste liquids.

M3. The method according to any of the preceding method embodiments, wherein the method further comprises
introducing a degradation unit into the usable space of the device.

M4. The method according to the preceding method embodiment, wherein the degradation unit is introduced into the usable space before heating.

M5. The method according to any of the 2 preceding method embodiments, wherein inserting the degradation unit into the usable space comprises electrically connecting the degradation unit to the device.

M6. The method according to any of the 3 preceding method embodiments, wherein the degradation unit is designed to decompose the active substance.

M7. The method according to any of the preceding method embodiments, wherein heating the usable space to the process temperature comprises increasing the process temperature until the relative humidity at the process temperature is below the upper humidity limit value.

M8. The method according to any of the preceding method embodiments, wherein heating the usable space to the process temperature comprises heating the usable space to an initial value for the process temperature, wherein the initial value is provided by the user.

M9. The method according to the preceding method embodiment, wherein the initial value is at least 37° C.

M10. The method according to any of the 2 preceding method embodiments, wherein, during and/or after heating to the initial value, the relative humidity in the usable space is determined and is compared with the upper humidity limit value.

M11. The method according to the preceding method embodiment, wherein, if the relative humidity is not below the upper humidity limit value, the process temperature is increased until the relative humidity is below the upper humidity limit value.

M12. The method according to method embodiment M7 or M11, wherein the process temperature is increased in stages until there is a drop below the upper humidity limit value.

M13. The method according to method embodiment M7 or M11, wherein increasing the process temperature comprises determining the process temperature based on the current relative humidity and temperature using the vapor pressure curve.

M14. The method according to any of the preceding method embodiments, wherein the process temperature is greater than or equal to the initial value and is selected such that the relative humidity at the process temperature is below the upper humidity limit value.

M15. The method according to any of the preceding method embodiments, wherein the process temperature is at least 37° C.

M16. The method according to any of the preceding embodiments, wherein the process temperature is at most 75° C., preferably at most 65° C., more preferably at most 55° C.

M17. The method according to any of the preceding method embodiments, wherein the relative humidity is not regulated during heating.

M18. The method according to any of the preceding method embodiments, wherein the upper humidity limit value is in the range of 45% to 75% relative humidity, preferably in the range of 45% to 60% relative humidity, more preferably in the range of 45% to 55% relative humidity.

M19. The method according to any of the preceding method embodiments, wherein the process temperature after heating is kept constant at least until the active substance in the usable space has degraded.

M20. The method according to any of the preceding method embodiments, wherein the method further comprises:
increasing the relative humidity in the usable space to a value above a lower humidity limit value.

M21. The method according to the preceding embodiment,
wherein the relative humidity in the usable space is increased to a value above a lower humidity limit value before the relative humidity is regulated to a first target value for a first duration.

M22. The method according to any of the 2 preceding embodiments,
wherein the relative humidity in the usable space is increased to a value above a lower humidity limit value after the usable space has been heated to the process temperature.

M23. The method according to any of the 3 preceding embodiments,
wherein increasing the relative humidity in the usable space to a value above a lower humidity limit value comprises vaporizing water.

M24. The method according to any of the 4 preceding embodiments, wherein the lower humidity limit value is in the range of 30% to 40% relative humidity.

M25. The method according to any of the 5 preceding embodiments, wherein the relative humidity in the usable space is increased to a value above a lower humidity limit value before the relative humidity in the usable space is regulated to a first target value.

M26. The method according to any of the preceding method embodiments, wherein the first target value is a first amount above the humidity at the start of regulating the relative humidity to a first target value.

M27. The method according to the preceding method embodiment, wherein the first amount is in the range of 10% to 40% relative humidity, more preferably in the range of 15% to 30% relative humidity, even more preferably in the range of 15% to 25% relative humidity.

M28. The method according to any of the preceding method embodiments, wherein the first duration is in the range of 5 min to 120 min, preferably in the range of 30 min to 90 min, more preferably in the range of 40 to 70 min.

M29. The method according to any of the preceding method embodiments, wherein introducing the active substance comprises vaporizing or nebulizing an active substance liquid in the humidifier of the humidification system.

M30. The method according to any of the preceding method embodiments, wherein the active substance liquid is provided in an active substance container and/or at an active substance port of the device.

M31. The method according to any of the preceding method embodiments, wherein the active substance liquid is an active substance solution.

M32. The method according to any of the preceding method embodiments, wherein the active substance is a sterilizing agent.

M33. The method according to any of the preceding method embodiments, wherein the active substance is hydrogen peroxide (H₂O₂).

M34. The method according to any of the preceding method embodiments, wherein the method further comprises:
after the first duration has elapsed,
determining the current relative humidity in the usable space, and
regulating the relative humidity to a second target value for a second duration, wherein the humidification system introduces an active substance liquid.

M35. The method according to the preceding method embodiment, wherein the second target value is a second amount above the current relative humidity.

M36. The method according to the preceding method embodiment, wherein the second amount is in the range of 10% to 40% relative humidity, more preferably in the range of 15% to 30% relative humidity, even more preferably in the range of 15% to 25% relative humidity.

M37. The method according to any of the 3 preceding method embodiments, wherein the second duration is in the range of 5 min to 120 min, preferably in the range of 30 min to 90 min, more preferably in the range of 40 to 70 min.

M38. The method according to any of the preceding method embodiments, wherein the steps of deactivating the regulation, degrading the active substance, and flushing are only performed after the first and/or second duration has elapsed.

M39. The method according to any of the preceding method embodiments, wherein regulating the relative humidity in the usable space to a first target value and/or a second target value comprises actively distributing the air in the usable space of the device.

M40. The method according to any of the preceding method embodiments and with the features of M29, wherein the method comprises diluting the active substance liquid prior to vaporization or nebulization.

M41. The method according to any of the preceding method embodiments and with the features of M3, wherein degrading the active substance in the usable space comprises activating the neutralization unit.

M42. The method according to any of the preceding method embodiments, wherein degrading the active substance comprises decomposing the active substance.

M43. The method according to any of the preceding method embodiments, wherein the degradation duration is selected such that a residual concentration of the gaseous active substance in the usable space does not pose a hazard to a user.

M44. The method according to any of the preceding method embodiments, wherein the degradation duration is in a range of 10 min to 600 min, preferably in a range of 20 min to 400 min, more preferably in a range of 30 min to 240 min.

M45. The method according to any of the preceding method embodiments, wherein the method further comprises: flushing at least some of the supply pipes that supply to a humidifier of the humidification system.

M46. The method according to the preceding embodiment, wherein flushing is performed bidirectionally.

M47. The method according to any of the 2 preceding method embodiments, wherein flushing comprises emptying liquid pipes into a waste container.

M48. The method according to the preceding method embodiment, wherein flushing comprises filling the emptied liquid pipes with fresh water.

M49. The method according to the preceding method embodiment, wherein flushing comprises emptying the liquid pipes into the waste container again.

M50. The method according to any of the 5 preceding method embodiments, wherein emptying and/or filling is performed by means of a bidirectional pump.

M51. The method according to any of the preceding embodiments, wherein the method further comprises: locking a door to the usable space of the device, wherein the locking is performed prior to heating the usable space to a process temperature.

M52. The method according to any of the preceding method embodiments, wherein the method further comprises: performing an automatic check of the device, wherein the check is performed prior to heating the usable space to a process temperature, and heating the usable space to a process temperature is only performed if the check is successful.

M53. The method according to the preceding embodiment, wherein performing the automatic check comprises:
checking a temperature control system,
checking a humidity system,
checking a ventilation system, and/or
checking a water quantity in a storage tank.

M54. The method according to any of the 2 preceding embodiments with the features of embodiment M3, wherein performing the automatic check comprises:
checking a status of the degradation unit.

M55. The method according to any of the 3 preceding embodiments, wherein the humidification system comprises a liquid sensor, wherein performing the automatic check comprises:
checking a status of the liquid sensor.

M56. The method according to any of the preceding embodiments, wherein, at least during the first duration, an alarm system that monitors at least one function of the device is active.

M57. The method according to any of the preceding method embodiments, wherein the device is a laboratory device.

M58. The method according to any of the preceding method embodiments, wherein the device is a climate control or heating device.

M59. The method according to any of the preceding method embodiments, wherein the device is an incubator.

Reference is made below to device embodiments. These embodiments are indicated by a D followed by a number. When device embodiments or D-embodiments are mentioned below, these embodiments are meant.

D1. A device, comprising
a usable space;
an active humidification system designed to increase the relative humidity in the usable space;
a humidity sensor for measuring the relative humidity in the usable space;
a temperature control system designed to increase the temperature in the usable space;
a temperature sensor for measuring the temperature in the usable space;
wherein the humidification system comprises at least one active substance port designed for connection to an active substance container containing an active substance liquid comprising an active substance; and
wherein the device is designed to introduce the active substance into the usable space via the humidification system.

D2. The device according to the preceding device embodiment, wherein the humidification system further comprises at least one waste port designed for connection to a waste container, wherein the device is designed to flush at least part of the humidification system and to direct resulting waste liquid to the waste port.

D3. The device according to any of the preceding device embodiments, wherein the humidification system comprises a humidifier designed to vaporize and/or nebulize liquids.

D4. The device according to the preceding device embodiment, wherein the humidifier is a vaporizer and/or a nebulizer.

D5. The device according to any of the preceding device embodiments, wherein the humidification system comprises a water port designed for connection to a water reservoir that provides fresh water.

D6. The device according to any of the preceding device embodiments, wherein the humidification system comprises a pump.

D7. The device according to the preceding device embodiment, wherein the pump is a bidirectional pump.

It will be understood that a bidirectional pump can pump in both directions.

D8. The device according to any of the 2 preceding device embodiments and with the features of embodiment D3, wherein the pump is designed to convey liquids from the active substance port to the humidifier.

D9. The device according to any of the 3 preceding device embodiments and with the features of embodiments D3 and D5, wherein the pump is designed to convey liquids from the water port to the humidifier.

D10. The device according to any of the preceding device embodiments, wherein the humidification system comprises a valve matrix designed to enable fluid connections between different ports.

D11. The device according to the preceding device embodiment, wherein the valve matrix is further designed to adjust the flow rate for at least one fluid connection.

D 12. The device according to any of the 2 preceding device embodiments and with the features of D6, wherein the valve matrix is arranged between the pump and the active substance port.

D13. The device according to any of the 3 preceding device embodiments and with the features of D5 and D6, wherein the valve matrix is arranged between the pump and the water port.

D14. The device according to any of the 3 preceding device embodiments and with the features of D2 and D6, wherein the valve matrix is arranged between the pump and the waste port.

D15. The device according to any of the 5 preceding device embodiments, wherein the valve matrix comprises at least 2 valves.

D16. The device according to any of the preceding device embodiments, wherein the device additionally comprises an air distribution system designed to circulate the air in the usable space.

D17. The device according to the preceding device embodiment, wherein the air distribution system comprises a fan.

D18. The device according to any of the preceding device embodiments, wherein the device further comprises a ventilation system designed to supply the usable space with supply air.

D19. The device according to any of the preceding device embodiments, wherein the device is designed to carry out the method according to any of the preceding method embodiments.

D20. The device according to any of the preceding device embodiments, wherein the device comprises a processing unit designed to control the device.

D21. The device according to the preceding device embodiment, wherein the processing unit is designed to carry out the method according to any of the preceding method embodiments.

D22. The device according to any of the preceding device embodiments, wherein the active substance liquid is an active substance solution.

D23. The device according to any of the preceding device embodiments, wherein the active substance is a sterilizing agent.

D24. The device according to any of the preceding device embodiments, wherein the active substance is hydrogen peroxide (H₂O₂).

D25. The device according to any of the preceding device embodiments, wherein the device is a laboratory device.

D26. The device according to any of the preceding device embodiments, wherein the device is a climate control or heating device.

D27. The device according to any of the preceding device embodiments, wherein the device is an incubator.

D28. The device according to any of the preceding device embodiments with the features of embodiments D3 and D6, wherein the humidification system further comprises a liquid sensor arranged between the humidifier and the pump.

M60. The method according to any of the preceding method embodiments, wherein the device is a device according to any of the preceding device embodiments.

Reference is made below to system embodiments. These embodiments are indicated by an S followed by a number. When system embodiments or S-embodiments are mentioned below, these embodiments are meant.

S1. A system, comprising
a device according to any of the preceding device embodiments; and
a degradation unit designed to degrade an active substance in the usable space of the device.

S2. The system according to the preceding system embodiment, wherein the degradation unit is designed to be placed in the usable space and electrically connected to the device.

S3. The system according to any of the preceding system embodiments, wherein the degradation unit comprises a UV lamp.

S4. The system according to any of the preceding system embodiments, wherein the degradation unit comprises a reaction partner and/or catalyst for the active substance.

S5. The system according to the preceding system embodiment, wherein the degradation unit comprises a fan designed to direct air over the reaction partner and/or catalyst.

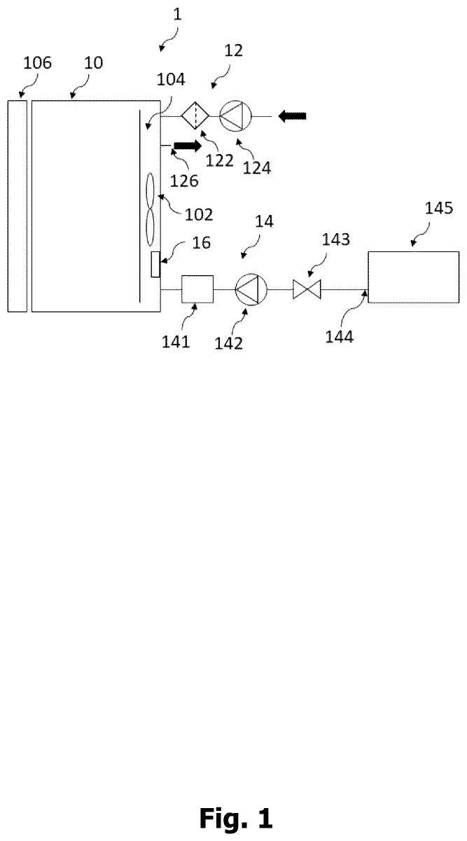

FIG. 1 shows a device 1 comprising a usable space 10, a humidification system 14, and a temperature control system 16, which are respectively designed to increase the relative humidity and the temperature in the usable space 10. In addition, the device 1 may also have, for example, a ventilation system 12, which is designed to supply the usable space 10 with supply air (e.g. ambient air). It will be understood that the device may also comprise other components; for example, the device 1 may additionally have a CO₂ control or regulation system that makes it possible to increase the CO₂ concentration in the usable space. The usable space may generally be accessible to a user outside the device via at least one door 106.

It will be understood that the invention relates in principle to a device 1 comprising a usable space 10, an active humidification system 14, and a temperature control system 16, such as, in general, a laboratory device, a climate control device, or a heating device. In particular, this may be an incubator.

The device 1 may further comprise an air distribution system, which may comprise a fan (or ventilator) 102 designed to circulate the air in the usable space 10. The fan 102 may preferably be arranged in an air duct 104.

For example, the temperature control system 16 may comprise a temperature control element, which is arranged in a corresponding air duct 104 and controls the temperature of the air flowing past, e.g. heats it or cools it. Alternatively, the temperature control element may also be mounted on the outside of an inner container which defines the usable space, and thus heats one or more walls of the usable space 10. For example, the temperature control element may be a heating element (e.g. a resistance heating element) or a Peltier system. The device 1 may also comprise a temperature sensor (not shown) which makes it possible to measure the temperature in the usable space. The temperature control system 16 can thus be controlled or regulated accordingly. It will be understood that the temperature control system 16 may also comprise a plurality of temperature control elements.

The humidification system 14 may comprise a humidifier 141, a pump 142, and a water port 144. The humidifier 141 may in particular comprise a nebulizer or vaporizer which can nebulize or vaporize liquid water and thus can increase the relative humidity of the air in the usable space. The water can accordingly be provided in a water reservoir 145, which in some embodiments is comprised by the device and in other embodiments is placed therein or is arranged externally thereto.

The water from the water reservoir 145 can then be conveyed by the pump 142 to the humidifier 141 via the water port 144, to which the water reservoir 145 can be connected. In addition, a first valve 143 may be provided, which is preferably arranged between the pump 142 and the water port 144 and can selectively interrupt the fluid connection therebetween.

The device may also comprise a humidity sensor (not shown) for measuring the relative humidity in the usable space. This enables the relative humidity in the usable space to be controlled or regulated by means of the humidification system 14 as a function of the measured relative humidity.

The optional ventilation system 12 may comprise, for example, an (air) pump 124 that can convey supply air (e.g. ambient air) into the usable space 10. Preferably, the ventilation system 12 may also comprise a filter 122 that filters the supply air to prevent contamination of the usable space 10. The filter may be a membrane filter, for example. The device may additionally comprise a pressure equalization opening 126, which may be part of the ventilation system. This has the advantage of preventing the pressure in the usable space from rising too much when supply air is actively being fed in. Preferably, the pressure equalization opening 126 has a flow resistance that hinders or reduces a constant exchange of air with the environment surrounding the device 1, so that such an exchange of air takes place primarily when there is a pressure difference between the usable space and the surrounding environment and is otherwise preferably strongly inhibited.

It will be understood that the pump 124 could alternatively also extract air from the usable space, thereby allowing supply air to flow in through the pressure equalization opening 126. In such embodiments in particular, the flow resistance can preferably be provided by a filter.

The present invention is based on the idea of using the humidification system 14 that already exists in a device 1 to introduce an active substance (preferably a sterilizing agent) into the usable space 10.

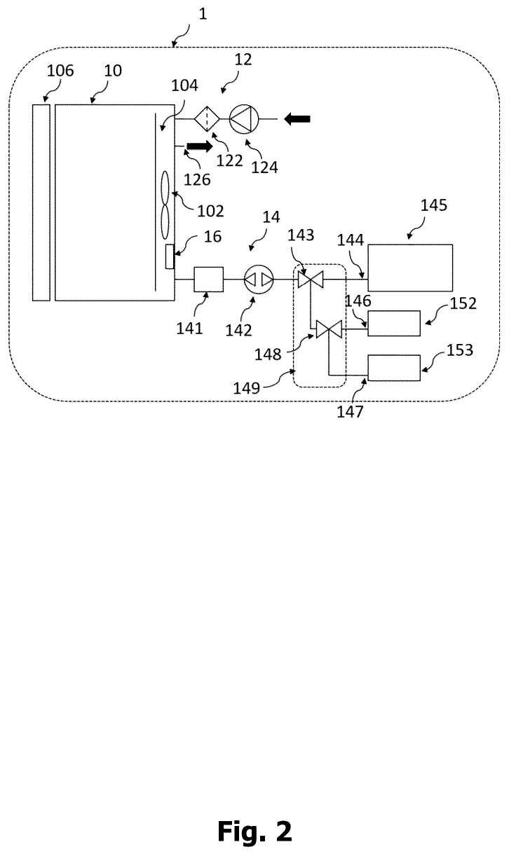

With reference to FIG. 2, it may be provided that the humidification system 14 comprises an active substance port 146 designed for connection to an active substance container 152. This active substance container 152 may comprise an active substance liquid, preferably a sterilizing agent, more preferably hydrogen peroxide. The humidification system may also comprise more than one active substance container port.

The active substance port 146 may be connected to the pump 142 via at least a first valve 143. For example, the first valve 143 may be a 3-way valve which can selectively connect the water port 144 or the active substance port 146 to the pump 142.

Furthermore, the humidification system may comprise a waste port 147 designed for connection to a waste container 153. The waste container 153 may be designed to receive waste liquids.

The waste container 153 may also be connected to the pump 142 via the first valve 143. Preferably, a second valve 148 may be provided, which for example can selectively connect the waste port 147 or the active substance port 146 to the first valve.

More generally, a valve matrix 149 may be provided, which can selectively fluidically connect the pump 142 to the water port 144, the active substance port 146, and the waste port 147.

Preferably, the pump 142 may be bidirectional. This can enable, in particular, effective flushing of at least some of the supply pipes.

Optionally, a liquid sensor may be provided between the pump 142 and the vaporizer 141; the liquid sensor is located as close to the vaporizer 141 as possible and is intended to detect the level of the liquid column during flushing. It is intended to compensate for fluctuations in the delivery rate of the pump 142 and ensure that a hose connecting the vaporizer 141 to the pump 142 is always filled with flushing water up to a defined point.

Overall, the device can thus also be designed to convey liquids from more than one container and/or reservoir simultaneously. In other words, the device 1 and in particular the humidification system 14 may be designed such that liquids can be conveyed by the pump 142 via several ports simultaneously; for example, liquid can be conveyed simultaneously from the active substance port 146 and the water port 144. For example, an active substance liquid can be produced by mixing 2 or more liquids. In particular, however, a concentrated active substance solution can also be diluted with water. This advantageously makes it possible to provide more active substance in the same size of active substance container, or, for example, to provide the same amount of active substance in a smaller active substance container 152.

In general, the present invention may comprise, in particular, a system which comprises, in addition to the device 1, also a degradation unit which is designed to degrade or decompose the active substance in the usable space 10 of the device 1. The degradation unit may also be referred to as a neutralization unit or decomposition unit.

The degradation unit can be placed in the usable space 10 and electrically connected to the device 1. For example, the degradation unit may comprise a UV lamp which can bring about the decomposition of the active substance (e.g. hydrogen peroxide) by means of UV radiation so that, once the germ density in the usable space has been reduced, a safe atmosphere can be created in the usable space before a user opens the door 106 to the usable space 10.

Preferably, the degradation unit may comprise a reaction partner or catalyst designed to degrade or decompose the active substance. More preferably, the degradation unit may comprise a fan that actively conveys air over the reaction partner or catalyst in order to increase efficiency. This has the advantage of providing a safe atmosphere in the usable space more quickly.

The device may also comprise a processing unit which may be designed to send data to the humidification system 14, the temperature control system 16, the air distribution system and/or the ventilation system 12 (and optionally also to receive data therefrom), to control or regulate them and/or to monitor them. In particular, the processing unit 15 may be connected to the humidification system 14 and the temperature control system 16. Such connections may be wired or wireless. Furthermore, the processing unit 15 may be connected to at least one sensor, which is arranged in such a way that it can provide a signal indicative of at least one parameter within the usable space 10. For example, the at least one sensor may be arranged within the usable space 10. In particular, the at least one sensor may be the humidity sensor and/or the temperature sensor.

The processing unit may be designed to regulate at least one variable (or parameter) for the conditions within the usable space 10. In particular, the processing unit may be designed to regulate the humidification system 14 and the temperature control system in such a way that a specified setpoint value is achieved for at least one parameter, in particular a temperature and/or relative humidity.

The processing unit may also be designed to control a degradation unit electrically connected to the device.

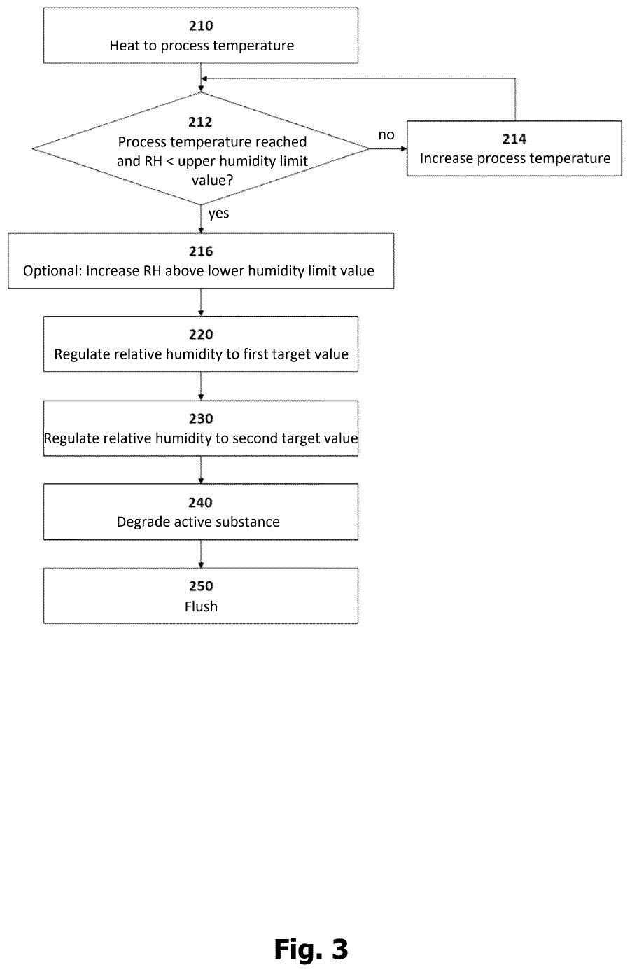

The present invention also relates to a method for reducing the germ density in the usable space of a device. With reference to FIG. 3, the method comprises heating the usable space to a process temperature (step 210). The process temperature preferably may initially correspond to an initial value, which more preferably may correspond to a temperature set by the user for subsequent processes, e.g. an incubation temperature, and preferably is at least 37° C. At this point, humidity regulation in the usable space is switched off, i.e. in particular the humidity is not actively being increased or maintained at a specific value.

Once the process temperature (in particular the initial value) has been reached (or even during heating), the relative humidity in the usable space can be determined and compared with an upper humidity limit value (step 212). If the relative humidity is above the upper humidity limit value, the process temperature is increased in order to further reduce the relative humidity (step 214). The adjusted process temperature can be calculated, for example, based on the current relative humidity and temperature using the vapor pressure curve. Alternatively, the process temperature can be increased in stages and the relative humidity checked until it is below the upper humidity limit value.

In embodiments of the invention, it may be provided that a lower humidity limit value is used. For example, the process temperature could be 40° C., and at this temperature the relative humidity (in step 212) could be 30%. If the upper humidity limit value is 50%, for example, no further takes place in step 214. In addition, it may be provided that a lower humidity limit value is used. For example, this could be 40% in the example. As described, the relative humidity in the example is 30% without further measures. If this is below the lower humidity limit value (40% in the example), the relative humidity can be increased in step 216 by vaporizing water until the relative humidity is above the lower humidity limit value. Using a lower humidity limit value can have the advantage of making the process exhibit more comparable relative humidity measurements and also become more comparable in terms of biological effectiveness.

Overall, the usable space is accordingly heated to a process temperature such that the relative humidity in the usable space is below the upper humidity limit value (steps 210, 212, 214). Optionally, it may be provided that the relative humidity in the usable space is above a lower humidity limit value (step 216). The final process temperature is then kept constant over the course of the entire method. The relative humidity, achieved as described (by setting the final process temperature and optionally vaporizing water if a lower humidity limit value is used), is used as the starting humidity in the method.

The present method can thus advantageously be carried out at 37° C. or slightly higher temperatures (e.g. 50° C.), so that no cooling time or at least no significant cooling time is required until the device can be used for other processes once the method has ended. Inter alia, the present method can thus advantageously be carried out faster than a typical 180° C. sterilization routine. In addition, the requirements for the materials for the overall system are lower, which widens the choice of materials since these do not need to be resistant to 180° C. Similarly, the insulation thickness of the device can advantageously be reduced compared to a device designed for corresponding heat sterilization.

The lower surface temperature inside the device when carrying out the method also advantageously makes it possible to treat materials that are not designed for temperatures in the region of 180° C., thus broadening the sterilization possibilities. Similarly, such a method can be advantageous for cleanroom applications, since the risk of increased particle release and disruption of the cleanroom air flow due to thermal effects is significantly lower compared to sterilization at 180° C.

Adjusting the process temperature such that the relative humidity is below an upper humidity limit value also advantageously makes it possible to automatically adapt the process to ambient conditions at the installation site, since the process temperature is adjusted as a function of the relative humidity. This means, for example, that the method can also be used in tropical areas with high humidity without any special adjustments.

Once the process temperature has been stabilized and the relative humidity, i.e. the starting humidity, is below the upper humidity limit value (and optionally above a lower humidity limit value), the relative humidity in the usable space is regulated to a first target value for a first duration (step 220). During this, the humidification system introduces a provided active substance, preferably H₂O₂, into the usable space. In particular, introducing the active substance may comprise vaporizing or nebulizing (e.g. atomizing) an active substance liquid, preferably an H₂O₂ solution. The first target value may preferably be a first amount above the starting humidity, i.e. the current relative humidity. Preferably, this first amount is in the range of 10% to 40% relative humidity, more preferably in the range of 15% to 30% relative humidity, even more preferably in the range of 15% to 25% relative humidity.

By using the sterilizing liquid to increase the relative humidity, an active substance (preferably H₂O₂) contained therein is released in the usable space. This can then reach relevant surfaces and reduce the germ density (and thus the germ load). Furthermore, an appropriate fan or air distribution system in the device can circulate the air in the usable space and thus evenly distribute the active substance. Once the relative humidity has reached the first target value, it is kept constant for the first duration.

Optionally, the active substance liquid may also be mixed from at least two different containers and/or reservoirs. In particular, a highly concentrated active substance solution may be diluted with water, for example. For this purpose, a plurality of containers and/or reservoirs may be simultaneously connected via one or more valves, e.g. a valve matrix, to a pump that conveys these liquids to the humidifier.

The present method advantageously requires just one sensor for the relative humidity in the usable space, which is typically already present when a humidification system is provided. In particular, there is no need for a sensor that can be used also to detect the active substance concentration. Rather, the method can be regulated and/or controlled solely via the humidity sensor.

After the first duration has elapsed, the above method can optionally (if necessary) be repeated. In particular, the current relative humidity in the usable space can be determined and then the relative humidity can be regulated to a second target value for a second duration (step 230). This can advantageously further reduce the germ density and, for example, enable sterilization of the surfaces in the usable space.

The second target value may also preferably be a second amount above the current relative humidity. As before, this second amount is preferably in the range of 10% to 40% relative humidity, more preferably in the range of 15% to 30% relative humidity, even more preferably in the range of 15% to 25% relative humidity. The first amount and the second amount may preferably be identical.

Subsequently, i.e. after the first duration or the second duration has elapsed, the humidity regulation is deactivated and the active substance in the usable space is degraded (step 240). Preferably, the active substance is degraded by means of a degradation unit. The degradation unit may be introduced into the usable space of the device at the start of the method and electrically connected thereto. This has the advantage of enabling the active substance to be degraded to such an extent that a residual concentration of the gaseous active substance that is harmless to users is achieved. For this purpose, the degradation unit may preferably be time-controlled.

Once the active substance has been degraded, the supply pipes to a humidifier (e.g. vaporizer) of the humidification system can be at least partially emptied into a waste container and flushed, in particular bidirectionally (step 250). This means that fresh water can be pumped from a corresponding reservoir to the humidifier and then into the waste container. This procedure may be repeated multiple times in order to remove residues of the active substance liquid in the supply pipes of the humidifier.

Finally, the waste container and the active substance container can be disposed of, and the device can be used again in normal operation.

The processing unit of the device may preferably be designed to carry out the method for reducing germ density.

The present invention thus advantageously uses existing components of devices to make it possible to reduce the germ density. More specifically, an existing humidification system can advantageously be extended to enable reduction of germ density and, in particular, disinfection and sterilization. Using parts of the humidification system may advantageously also make it possible that at least part of the pipe system, which in normal operation is used for water, is decontaminated using liquid sterilizing agent, and the germ density in the parts of the pipe system is reduced accordingly.

Overall, the present method advantageously enables an automated and controlled process that can be regulated via the device (e.g. the incubator).

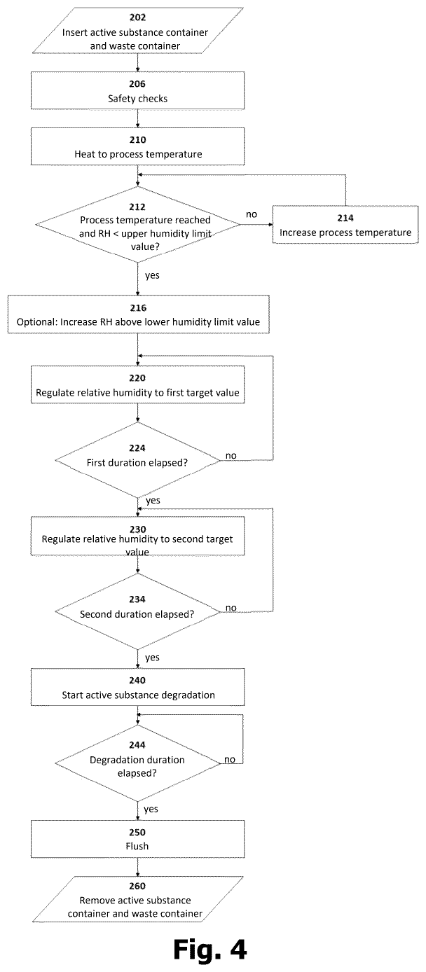

FIG. 4 shows a somewhat more detailed flow chart of an exemplary embodiment of the method for reduce the germ density in the usable space of a device. The method will be described below with regard to sterilizing the surfaces in the usable space, but it will be understood that simply a reduction in the germ density or disinfection of the usable space may also take place, i.e. sterilization is not mandatory.

At the beginning, the user inserts an active substance container and a waste container into the device (step 202). In other words, the user may insert a cartridge containing a liquid active substance (including sterilizing agents), such as hydrogen peroxide, and may connect a waste cartridge for liquids. The active substance container may typically contain a solution comprising the active substance (preferably H₂O₂) and water. In addition, it may be provided that the user introduces a degradation unit (also known as a decomposition unit or neutralization unit) into the usable space of the device and electrically connects it to this device. The degradation unit may in principle be designed to render the active substance harmless for a user (e.g. to neutralize and/or decompose it). For example, the degradation unit may comprise or consist of a UV lamp, or alternatively may comprise a fan with a reaction partner for the active substance or a catalyst in order to actively convey the air over the latter.

It may then be provided that safety checks are carried out (step 206). For example, it may be checked that the usable space of the device is closed and secured against unauthorized opening, and/or that the degradation unit has been placed in the usable space and connected. In addition or as an alternative, it may be checked whether other device openings are closed (e.g. access openings that are accessible to the user), whether an internal device fan is functioning, and/or whether an optional liquid sensor between the pump 142 and the vaporizer 141 is functioning.

The process of conditioning the usable space for sterilization can then begin. In a first step, the usable space and, in particular, the air in the usable space is heated to an initial value (i.e. to an initial process temperature) (step 210). This is at least 37° C. and can be specified by the user (e.g. the initial process temperature can correspond to an incubation temperature selected by the user). During heating, the humidity regulation is switched off. During heating or once the process temperature has been reached, the current humidity (i.e. the relative humidity) in the usable space can be determined and compared with the upper humidity limit value (step 212). The upper humidity limit value may be, for example, 55% relative humidity. If the humidity is below the upper humidity limit value, the process temperature remains as initially set.

If the relative humidity is not below the upper humidity limit value, i.e. the upper humidity limit value is exceeded, the process temperature is increased accordingly in order to reduce the relative humidity in the usable space to below the upper humidity limit value (step 214). The process temperature can be calculated from the measured value of the current relative humidity and temperature using the vapor pressure curve, or the process temperature can be increased in stages and the relative humidity checked until it is below the upper humidity limit value.

Reducing the relative humidity to below the corresponding upper humidity limit value serves to ensure that there is a required margin to the saturation limit, since the relative humidity in the usable space will increase as the active substance is introduced (e.g. by vaporizing an active substance solution), and saturation of the air is to be avoided.

In this embodiment, it may optionally also be provided that a lower humidity limit value is used, i.e. it is checked that the relative humidity is above a lower humidity limit value. This could be 30% or 40%, for example, with a lower humidity limit value of 40% being assumed in the following example. If, for example, a temperature of 40° C. is set in step 210 and this results in a relative humidity of 30% (with an upper humidity limit value of 55% and a lower humidity limit value of 40%), this does not lead to any further increase in the process temperature in step 214. However, the relative humidity can be increased in step 216 by vaporizing water. The humidity thus reached is the starting humidity in this example.

The relative humidity at the start of step 220 (i.e. after completion of step 212, which may have been carried out multiple times, and after the optional step 216) is the starting humidity, and the corresponding process temperature is kept constant for the remainder of the method.

The actual sterilization (or, more generally, reduction of the germ density) can then begin by regulating the relative humidity to a first target value (step 220). For this purpose, the liquid is conveyed from the active substance container (e.g. H₂O₂ cartridge) to the humidifier, which vaporizes or nebulizes it accordingly and thus introduces the active substance into the usable space. The first target value may be a (fixed) first amount (e.g. 15%-25% relative humidity) above the current relative humidity.

The active substance liquid (also sterilizing liquid) is vaporized (or nebulized), thereby increasing the humidity in the usable space. The active substance is thus released into the usable space and reaches the surfaces to be sterilized. The active substance can advantageously be evenly distributed in the usable space by means of an air distribution system of the device.

The relative humidity is then maintained at the first target value for a first duration (step 224), i.e. the relative humidity is kept constant.

After the first duration has elapsed, the process may be repeated to ensure sterilization or a log 12 reduction, for example. To do this, the current relative humidity is determined and then is regulated to a second target value (step 230). The second target value is again a (fixed) second amount (e.g. 15%-25% relative humidity) above the current relative humidity. This causes the concentration of the active substance in the usable space to increase again. The relative humidity is then maintained at the second target value for a second duration (step 234), i.e. the relative humidity is kept constant.

After the second duration has elapsed, the humidity regulation can be deactivated. The active substance degradation in the usable space is then started (step 240).

For this purpose, a degradation unit installed in the usable space can preferably be activated by a device controller for a degradation duration (i.e. a certain length of time) (step 244). The degradation duration can preferably be selected such that the residual concentration of the gaseous active substance does not pose any hazard to the user when the latter opens a door to the usable space, for example.

Subsequently, the corresponding liquid pipes to the humidifier (e.g. vaporizer) can be flushed (step 250). For this purpose, the liquid pipes can be emptied into the waste container and filled with fresh water from a water tank, which can then be pumped back into the waste container. This may be repeated multiple times.

Finally, the active substance container and the waste container can be removed and disposed of (step 260).

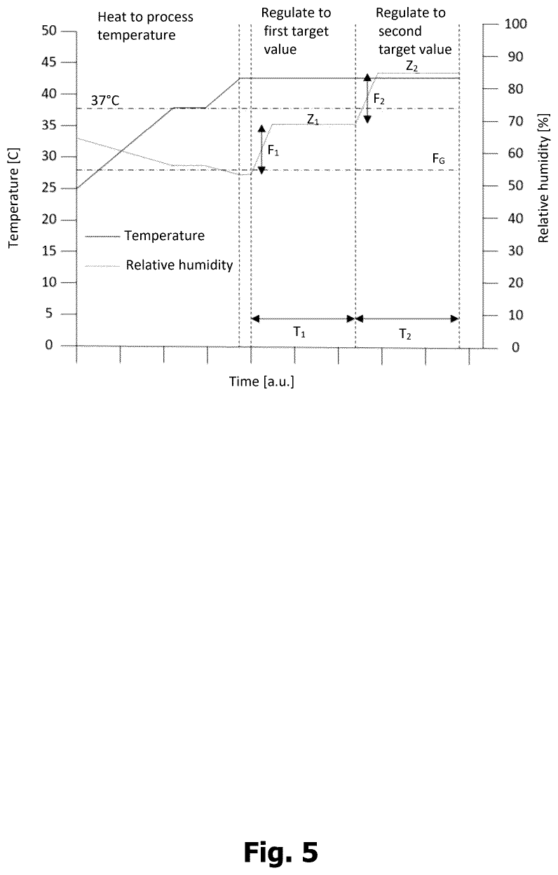

FIG. 5 illustrates an example curve of temperature and relative humidity in the usable space of a device when carrying out the method for reducing the germ density. It will be understood that this is a purely illustrative and schematic representation and that changes in temperature and/or relative humidity, for example, typically do not follow a purely linear course.

At the beginning, the air in the usable space is, for example, at 25° C. and approx. 65% relative humidity. The air is then heated to the process temperature. First, the temperature is heated to an initial value, which is at least 37° C. and corresponds to this value in the present example. Increasing the temperature in the usable space reduces the relative humidity. In the present example, once the initial value, i.e. 37° C., has been reached, it is checked whether the relative humidity is below a corresponding upper humidity limit value FG, which in the present example is 55% relative humidity. Alternatively, the relative humidity may, for example, also be compared with the upper limit value during heating. In the present case, the relative humidity is still just above the upper humidity limit value FG.

Therefore, the process temperature is adjusted and the usable space is accordingly heated further. After heating again to approx. 43° C., the relative humidity is below the upper humidity limit value F_{G}. Therefore, the actual reducing of the germ load can then begin. For this purpose, the relative humidity is regulated for a first duration T₁ to a first target value Z₁, which for example is a first amount F₁ above the current humidity (also the starting humidity). During this, an active substance liquid, preferably an active substance solution, is vaporized by means of the humidification system of the device. This enables the active substance to be introduced into the usable space.

After the first duration T₁ has elapsed, the relative humidity can be determined and again increased, for example to ensure a sufficient reduction in germ density for sterilization. In other words, the relative humidity can be regulated from the then current relative humidity to a second target value Z₂ for a second duration T₂: This second target value Z₂ may for example be a second amount F₂ above the current relative humidity. By increasing the relative humidity, active substance is again introduced into the usable space.

In the present example, the first duration T₁ is identical to the second duration T₂ and the first amount F₁ is identical to the second amount F₂. However, these may also be selected to be different from each other.

The active substance in the usable space can then be degraded and pipes of the humidification system can be flushed, as described above. During this, the temperature may advantageously continue to be kept constant in order to prevent condensation within the usable space. Due to the tightness of the device, the humidity typically does not drop during the degradation of the active substance.

The present invention thus makes it possible to use existing components of a humidification system also to reduce the germ density in the usable space. Advantageously, parts of the pipe system can also be cleaned with the active substance liquid used. In addition, high temperatures are not necessary, which on the one hand makes it easier to select materials for the device, reduces the required insulation thickness, and also makes it possible to treat objects that cannot tolerate high temperatures of approx. 180° C. Longer cooling phases are also no longer necessary, meaning that the device can be ready for use again more quickly.

The method can automatically adapt to the conditions at the installation site (in particular the relative humidity of the surrounding environment) and adjust the process temperature accordingly. Overall, this has the advantage of enabling the method to be carried out automatically via the device's own control system.

An exemplary process can proceed as follows:
1. A user inserts a cartridge containing liquid sterilizing agent (e.g. hydrogen peroxide) and connects a waste cartridge for liquids.

A degradation unit (e.g. a decomposition unit) is placed in the usable space and electrically connected to the device; it may comprise, for example:
a fan with a reaction partner for the sterilizing agent or with a catalyst in order to actively convey the air over the latter
a UV lamp.
2.1 Start of conditioning phase: Heating to sterilization temperature (incubation temperature set by the user, e.g. at least 37° C.), humidity regulation is switched off.
2.2 Monitoring the starting humidity

It will be understood that the humidity in the usable space increases during the vaporization in step 3. Before starting this vaporization, it can be ensured that there is a margin to the saturation limit. The vaporization cycle should be run twice. Against this background, it may be advantageous to select a relatively low starting humidity.

During heating or once the sterilization temperature has been reached, the current humidity in the usable space is compared with an upper limit value—this could be, for example, 55% relative humidity, i.e. 55% RH.

If the upper limit value is exceeded, the temperature is increased in the present case. This reduces the value of the relative humidity in the usable space. A new sterilization temperature can be determined. Possible variants for determining the new sterilization temperature are:
calculating the target temperature from the measured value of the current relative humidity and temperature using the vapor pressure curve, or
increasing the temperature value of the device in stages and checking the relative humidity again until it is below the starting humidity limit value.

If the value is below the upper limit value, the sterilization temperature remains as in 2.1.

Optionally, a lower humidity limit value may also be used. This could be 40% RH, for example. If the humidity value is below the lower humidity limit value, the relative humidity can be increased, for example by vaporizing water, until the lower humidity limit value is exceeded.

The starting humidity is therefore achieved by using an upper humidity limit value (and increasing the temperature as long as this limit value is exceeded) and optionally by using a lower humidity limit value (and vaporizing water as long as this limit value is not reached). The humidity thus achieved is the starting humidity.

The temperature determined as described (sterilization temperature) is kept constant by the incubator throughout the entire further process.
3. Start of sterilization process

The humidity regulation is activated and liquid is conveyed from the H₂O₂ cartridge. The H₂O₂ cartridge can also be referred to as a second container (where a first container contains water). The setpoint for the relative humidity may be a fixed amount (e.g. 15-25% RH) above the current relative humidity.

The sterilizing liquid is vaporized, thereby increasing the humidity in the usable space. The active substance is thus released into the usable space and reaches the surfaces to be sterilized. The substance is evenly distributed in the usable space by normal device ventilation. This humidity is kept constant.

After a specified time has elapsed, the sterilization process is repeated by measuring the current relative humidity again and increasing the setpoint by a fixed amount (e.g. 15-25% RH). This causes the concentration of the active substance in the usable space to rise again.

This humidity is again kept constant for a specified time.
4. Degradation of the sterilizing agent

After a sterilization time has elapsed, the humidity regulation is deactivated. The decomposition unit placed in the usable space is activated for a certain length of time by the device controller.

The time is selected such that the residual concentration of gaseous H₂O₂ does not pose any hazard to the user.
5. The liquid pipes to the vaporizer are emptied (into a waste container) and filled with fresh water from the water tank, which is then pumped back into the waste container. This may be repeated multiple times.
6. The cartridge containing the remaining sterilizing agent and the waste cartridge are disposed of.

When a relative term such as “about,” “substantially,” or “approximately” is used in this description or the claims, any such term should also be construed as also including the exact term. That is to say, for example, “substantially straight” should be construed as also including “(exactly) straight.”

Whenever steps have been recited above or also in the appended claims, it should be noted that the order in which the steps are recited in this text may be random. This means that the order in which the steps are recited may be random unless otherwise specified or obvious to a person skilled in the art. For example, when it is stated in the present document that a method comprises steps (A) and (B), this does not necessarily mean that step (A) takes place before step (B), but it is also possible that step (A) is carried out (at least in part) simultaneously with step (B), or that step (B) takes place before step (A). Furthermore, when it is stated that one step (X) precedes another step (Z), this does not mean that there is no step between steps (X) and (Z). That is, step (X) before step (Z) includes the situation where step (X) is carried out directly before step (Z), but also the situation where (X) is carried out before one or more steps (Y1), . . . , followed by step (Z). The same considerations apply when terms such as “after” or “before” are used.

While a preferred embodiment has been described above with reference to the accompanying drawings, the person skilled in the art will understand that this embodiment was provided for illustration only and should by no means be construed as limiting the scope of the present invention, which is defined by the claims.

What is claimed:

1. A method for reducing a germ density in the usable space of a device, comprising
heating the usable space to a process temperature such that the relative humidity in the usable space is below an upper humidity limit value;
regulating the relative humidity in the usable space to a first target value for a first duration, wherein a humidification system introduces an active substance;
deactivating the relative humidity regulation; and
degrading the active substance in the usable space for a degradation duration.

2. The method according to claim 1, wherein heating the usable space to the process temperature comprises increasing the process temperature until the relative humidity at the process temperature is below the upper humidity limit value.

3. The method according to claim 1, wherein the relative humidity is not regulated during heating.

4. The method according to claim 1, wherein the process temperature after heating is kept constant at least until the active substance in the usable space has degraded.

5. The method according to claim 1, wherein the method further comprises:
increasing the relative humidity in the usable space to a value above a lower humidity limit value.

6. The method according to claim 1, wherein the first target value is a first amount above the humidity at the start of regulating the relative humidity to a first target value.

7. The method according to claim 1, wherein introducing the active substance comprises vaporizing or nebulizing an active substance liquid in the humidifier of the humidification system.

8. The method according to claim 1, wherein the active substance is hydrogen peroxide (H₂O₂).

9. The method according to claim 1, wherein the method further comprises:
after the first duration has elapsed,
determining the current relative humidity in the usable space, and
regulating the relative humidity to a second target value for a second duration, wherein the humidification system introduces an active substance liquid, wherein the second target value is a second amount above the current relative humidity.

10. The method according to claim 1, wherein the method further comprises: locking a door to the usable space of the device, wherein the locking is performed prior to heating the usable space to a process temperature, and wherein the method further comprises: performing an automatic check of the device, wherein the check is performed prior to heating the usable space to a process temperature, and heating the usable space to a process temperature is only performed if the check is successful.

11. The method according to claim 1, wherein the device is a climate control or heating device or is an incubator.

12. A device, comprising
a usable space;
an active humidification system designed to increase the relative humidity in the usable space;
a humidity sensor for measuring the relative humidity in the usable space;
a temperature control system designed to increase the temperature in the usable space;
a temperature sensor for measuring the temperature in the usable space;
wherein the humidification system comprises at least one active substance port designed for connection to an active substance container containing an active substance liquid comprising an active substance; and
wherein the device is designed to introduce the active substance into the usable space via the humidification system.

13. The device according to claim 12, wherein the device is designed to perform a method comprising:
heating the usable space to a process temperature such that the relative humidity in the usable space is below an upper humidity limit value;
regulating the relative humidity in the usable space to a first target value for a first duration, wherein a humidification system introduces an active substance;
deactivating the relative humidity regulation; and
degrading the active substance in the usable space for a degradation duration

14. A system, comprising
a device according to claim 12; and
a degradation unit designed to degrade an active substance in the usable space of the device.
